(19) United States
(12) Patent Application Publication
(10) Pub. No.: US 2023/0405236 A1
(43) Pub. Date: Dec. 21, 2023

(54) INJECTION DEVICE

(71) Applicant: NEMERA SZCZECIN SPOLKA Z OGRANICZONA ODPOWIEDZIALNOSCIA, Szczecin (PL)

(72) Inventors: Mateusz WILCZEK, Szczecin (PL); Arkadiusz Lukasz WOITAS, Szczecin (PL); Alberto LOZANO PLATONOFF, Szczecin (PL)

(21) Appl. No.: 18/247,377

(22) PCT Filed: Oct. 1, 2021
(86) PCT No.: PCT/IB2021/059032

§ 371 Date: Mar. 30, 2023

(30) Foreign Application Priority Data
Oct. 1, 2020 (PL) P435617

(51) Int. Cl. A61M 5/315

(52) U.S. Cl. CPC A61M 5/31578; A61M 5/31541; A61M 5/31553

(57) ABSTRACT

An injection device comprising a housing (1) and a cartridge holder (2, 20), the housing (1) being provided on the proximal side with a rotatable dose knob (4, 40) and an axially translatable button (5, 50). The housing (1) comprises: a dose setting sleeve (6, 60), a non-rotatable tubular driving element (7, 70) slidingly mounted in the housing (1) and engaged with the dose setting sleeve (6, 60) by a threaded connection, a non-rotatable axially sliding piston rod (15,150), a toothed gear (8, 80) comprising teeth (8.1, 80.1), a driving spring (13, 130). The dose setting sleeve (6, 60) is equipped with at least one ratchet arm (6.4, 60.4) cooperating with the toothed gear (8, 80); the piston rod (15, 150) has at least one row of ratchet teeth (15.1, 150.1) on its external surface; the driving element (7, 70) is on its distal side fixedly connected with at least one driving arm (14, 140) cooperating with the ratchet teeth (15.1, 150.1) of the piston rod (15, 150). The injection device further comprises: a non-rotatable and axially slidable button sleeve (9, 90) engaged on its proximal side with the button (5, 50), an immovable auxiliary sleeve (10, 100) located within the driving element (7, 70), wherein the button sleeve (9, 90) is detachably and slidingly engaged with the toothed gear (8, 80) and the button sleeve (9, 90) is slidingly engaged with the auxiliary sleeve (10, 100) by means of a coupling spring (12, 120).

The present application relates to an injection device, in particular for injection of a liquid substance which is a medicament in a liquid form.

Devices for injecting a liquid substance, in particular a medicament in a liquid form by subcutaneous injection, are known in the art. Such devices may be designed for a one time injection (auto-injector) or they may enable repeated self-injections of a medication by a patient. Injection devices are specifically known as pen-injectors for repeated self-injections of a medication by a patient. They are particularly useful in case of illness requiring delivery of a medication on regular basis over a long period of time, in particular in diabetes type 1 and type 2. Other non-limiting examples of use of the injection devices include delivery of teriparatide in osteoporosis treatment, growth hormone and delivery of medicaments used in infertility treatment.

The injection devices usually comprise a mechanism for driving a piston rod, the mechanism being enclosed in a housing, and the piston rod being moved axially during injection of a dose and pressing a piston causing a required dose of a medicament to be pushed out of the cartridge. The mechanism for driving the piston rod and optionally for selecting a dose is enclosed in the housing which is connected with a cartridge holder receiving a medicament cartridge or alternatively the housing and the cartridge holder constitute an integral element.

In manual injectors the force necessary for axial movement of the piston rod is applied directly by a user. In order to deliver a dose a button is pressed, the button being usually located at the end of the injector and the axial movement of the button is converted into an axial movement of the piston rod by the mechanism of the injector. Examples of know manual injectors are disclosed in WO99/38554A1 and WO2004/078241A1.

Automatic injectors comprise an element for storing energy which is subsequently used for driving the piston rod during injection of a dose. This element is usually a spring which is preloaded by a user by means of a knob, the displacement of which is transferred to the spring by the mechanism of the injector. The deformation of the spring results in that energy is stored within. The devices of this type are disclosed for example in the publications WO2006/076921A1 and WO2012/154110A1. In the injector known from WO2013/144021A1, the spring is tensed during production so that the energy sufficient for delivery of the whole medicament contained in the cartridge is stored in the spring.

The injectors may enable to increase and optionally to reduce the volume of a set dose or to reset the device, i.e. to make it return to its initial position without delivering a dose.

The injectors may also be disposable, so that upon delivery of the whole volume of the medicament contained in the cartridge, in one or multiple injections, the cartridge may not be replaced, or they may be multi-use devices enabling installation of a new medicament cartridge.

The aim of the invention was to provide an injection device with a compression spring, the injection device being provided with a new mechanism for setting and optionally correcting the dose of the injected substance.

A compression spring has a more simple construction than the springs which are tensed by a rotational movement. In particular, a compression spring may be mounted directly between mutually movable elements of the injector, and it is also smaller than a torsional spring.

According to the invention an injection a device is provided for injecting a liquid substance, the injection device having a longitudinal axis, a proximal end and a distal end, and comprising a housing and a cartridge holder in which a cartridge with said substance is contained, the housing being provided on the proximal side with a rotatable dose knob and an axially translatable button, the housing comprising:
a dose setting sleeve adapted to be rotationally engaged with the dose knob
a non-rotatable tubular driving element slidingly mounted in the housing, the driving element being engaged with the dose setting sleeve by a threaded connection
a non-rotatable axially sliding piston rod adapted to press against a piston located in the cartridge
a toothed gear comprising teeth
a driving spring
   wherein
the dose setting sleeve is equipped with at least one ratchet arm cooperating with the toothed gear
the piston rod has at least one row of ratchet teeth on its external surface
the driving element is on its distal side fixedly connected with at least one driving arm cooperating with the ratchet teeth of the piston rod.

The injection device according to the invention is characterized in that it further comprises:
a non-rotatable and axially slidable button sleeve engaged on its proximal side with the button
an immovable auxiliary sleeve located within the driving element
   and in that the button sleeve is detachably and slidingly engaged with the toothed gear and the button sleeve is slidingly engaged with the auxiliary sleeve by means of a coupling spring which is compressed when the button sleeve is translated in the distal direction.

The dose setting sleeve may be adapted to be engaged with the dose knob by means of a tubular coupling element located between the dose setting sleeve and the button sleeve, the tubular coupling element being rotationally engaged with the dose knob and being provided at its distal end with at least one coupling arm cooperating with at least one ratchet arm of the dose setting sleeve; the at least one driving arm of the driving element may be movable in a direction perpendicular to the longitudinal axis of the device.

The button sleeve may be engaged at its distal end with a cam sleeve in a way enabling their mutual rotation, the cam sleeve cooperating with a cam element which is rotationally coupled with the cam sleeve, the cam element cooperating with at least one driving arm of the driving element so as to enable its/their displacement between a position of engagement with the ratchet teeth of the piston rod and a position of disengagement with the ratchet teeth of the piston rod.

The dose setting sleeve may be further provided at its distal end with at least one driving projection designed for being abutted by at least one coupling arm of the coupling element, so as to cause rotation of the dose setting sleeve.

The device may comprise at its distal end a piston rod guide enabling the piston rod to move in the distal direction and blocking the movement of the piston rod in the proximal direction.

The auxiliary sleeve may rest by its distal end on the piston rod guide.

The driving element may be blocked against rotation in the housing by means of a multi-spline.

The dose setting sleeve may be blocked in rotation with the dose knob by means of a multi-spline.

The coupling element may be blocked in rotation with the dose knob by means of a multi-spline.

The button sleeve may be provided with an external annular part fixedly connected therewith, the annular part comprising on its proximal side an internal multi-spline enabling a sliding detachable engagement with the toothed gear, and on is distal side an external multi-spline enabling a sliding engagement with the auxiliary sleeve.

The number of the coupling arms of the coupling element may equal the number of the ratchet arms and the number of the driving projections of the dose setting sleeve.

The teeth of the toothed gear may be arranged annularly.

The teeth of the toothed gear may be located internally or externally.

The teeth of the toothed gear may be mutually parallel.

The toothed gear may be blocked against rotation during dose setting and it may rotate during dose delivery.

The driving spring may be a compression spring, in particular a helical compression spring.

The mechanism of the disclosed injection device, in particular the drive mechanism of the piston rod, is simple and reliable because the force activating injection acts in the same direction as the piston is displaced.

The device has compact size and allows for delivery of a liquid substance, in particular a medicament in a liquid form, in the therapies requiring multiple injections of medicament doses the volume of which may be selected by a user, specifically it may be increased or also reduced, as well as in the therapies requiring multiple injections of the same doses of medicament.

Preferred embodiments are shown in the drawings in which:

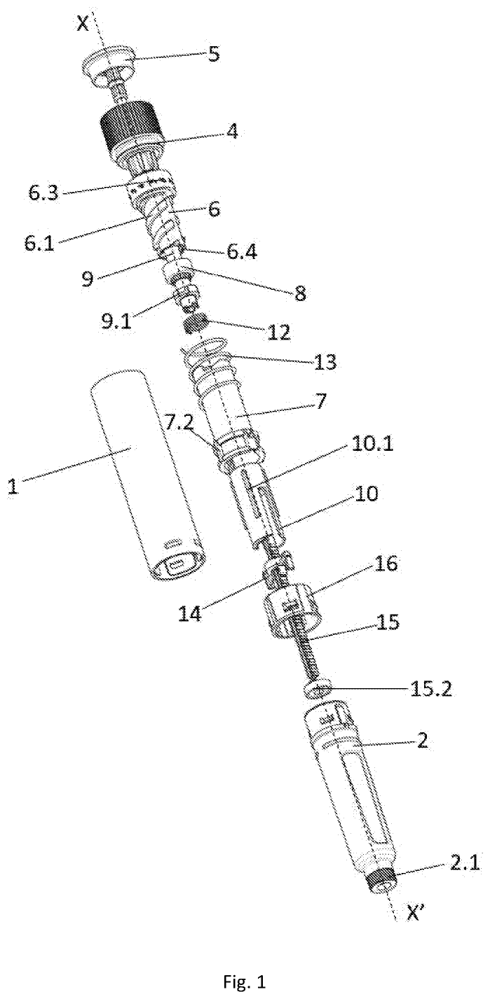

FIG. 1 shows an exploded view of a first embodiment of the injection device according to the invention;

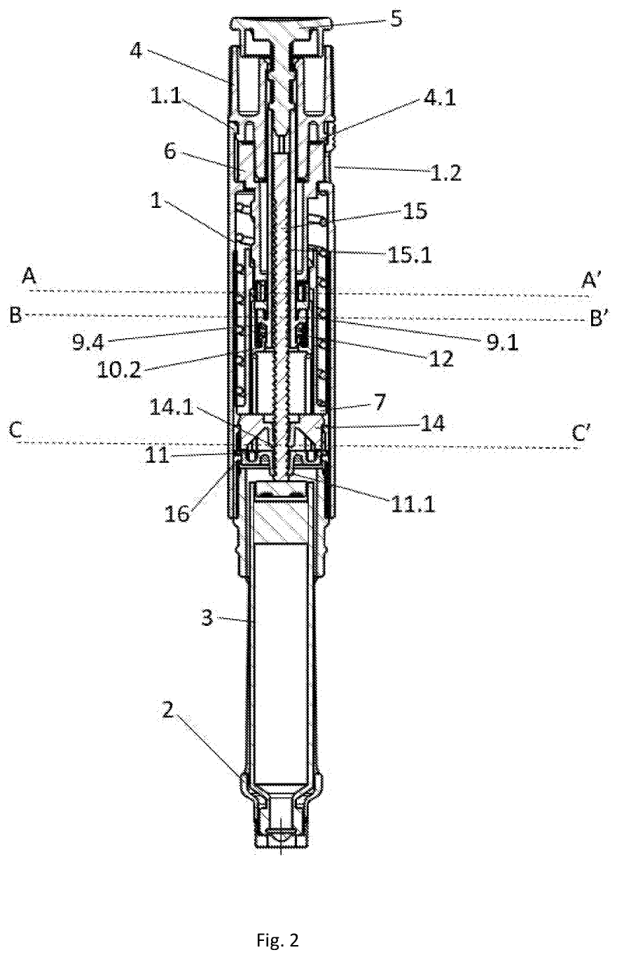

FIG. 2 shows a longitudinal sectional view of the first embodiment of the injection device according to the invention;

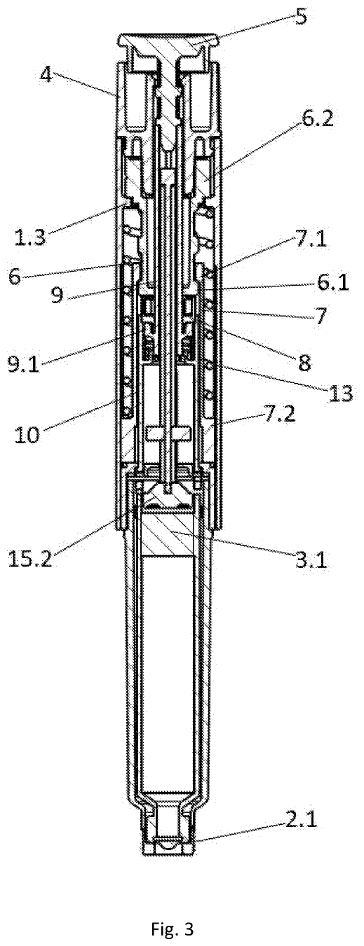

FIG. 3 shows another longitudinal sectional view of the first embodiment of the injection device according to the invention;

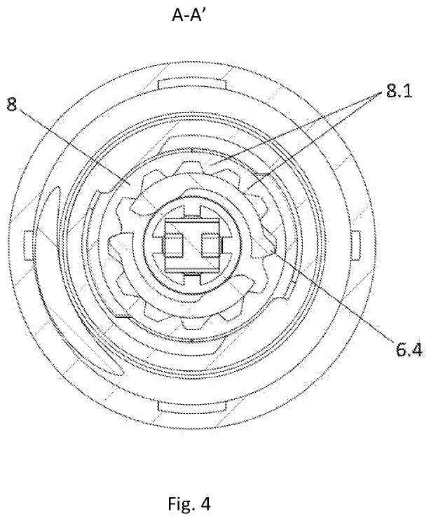

FIG. 4 shows a cross-sectional view along a plane A-A′ indicated in FIG. 2, of the first embodiment of the injection device according to the invention;

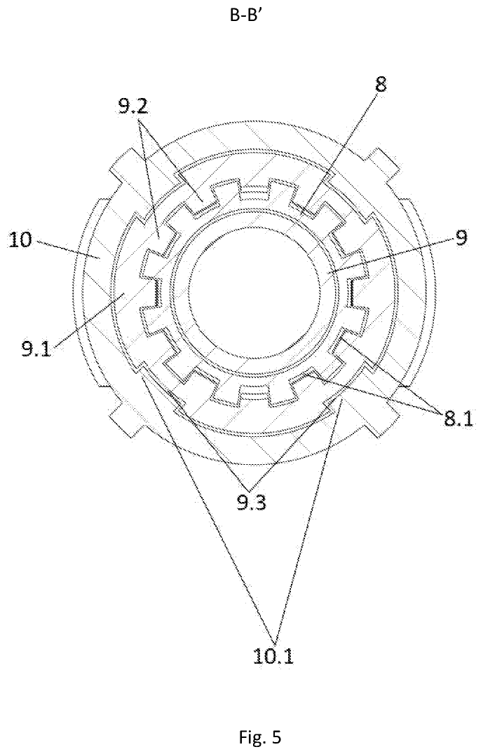

FIG. 5 shows a cross-sectional view along a plane B-B′ indicated in FIG. 2, of the first embodiment of the injection device according to the invention;

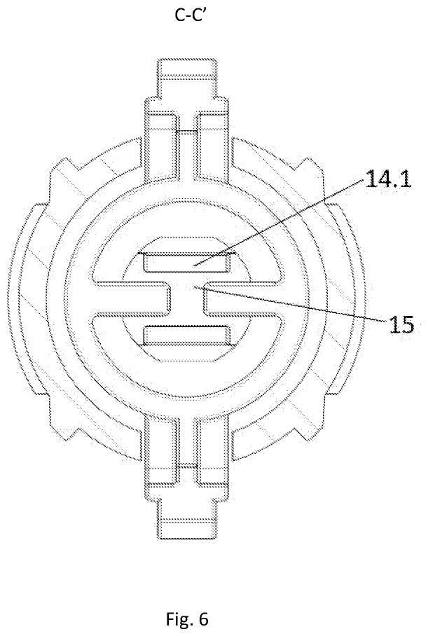

FIG. 6 shows a cross-sectional view along a plane C-C′ indicated in FIG. 2, of the first embodiment of the injection device according to the invention;

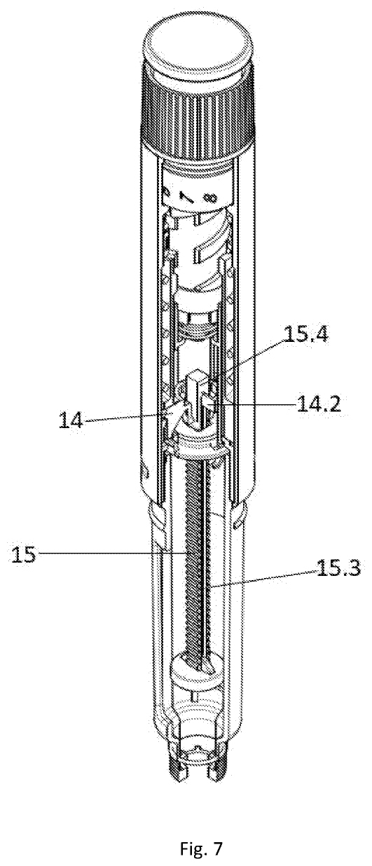

FIG. 7 shows a partial axonometric view of the first embodiment of the injection device according to the invention, showing a last dose blocking mechanism;

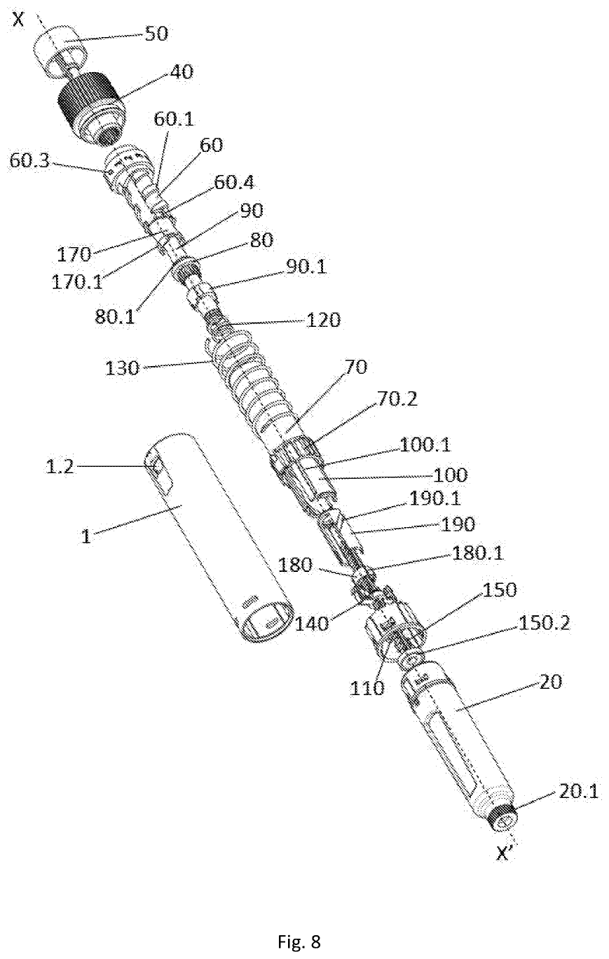

FIG. 8 shows an exploded view of a second embodiment of the injection device according to the invention;

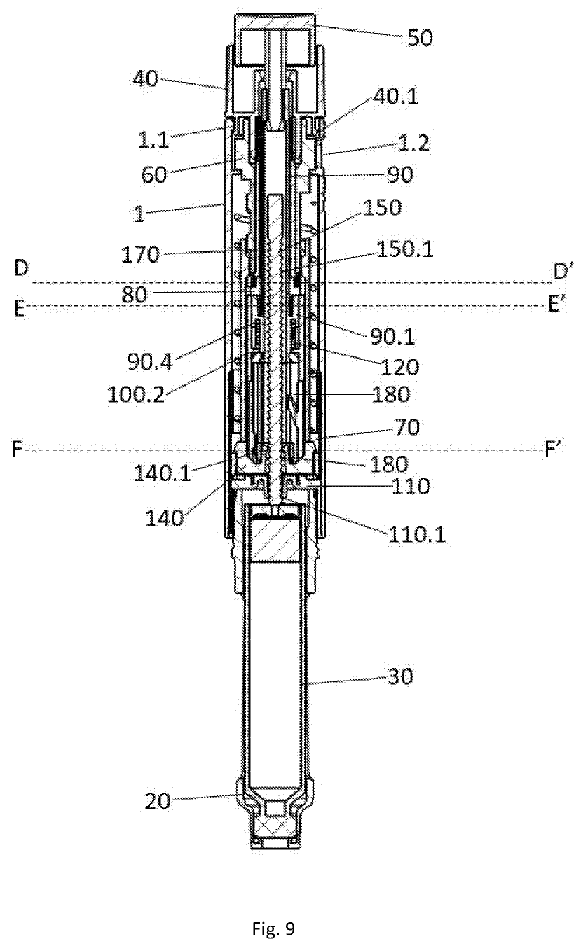

FIG. 9 shows a longitudinal sectional view of the second embodiment of the injection device according to the invention;

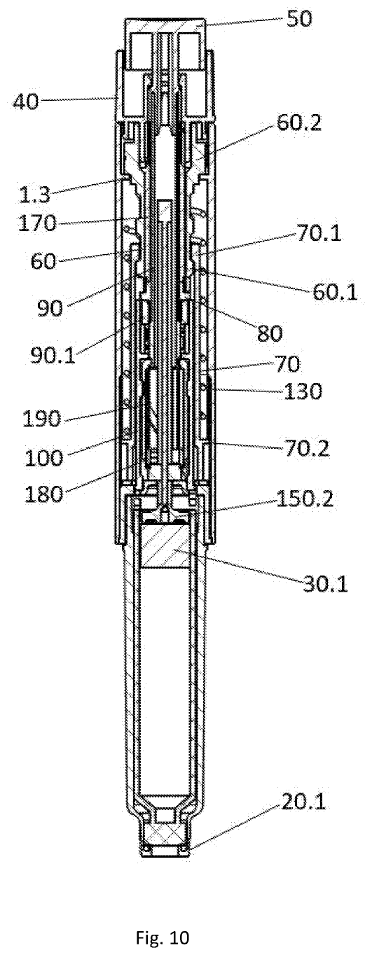

FIG. 10 shows another longitudinal sectional view of the second embodiment of the injection device according to the invention;

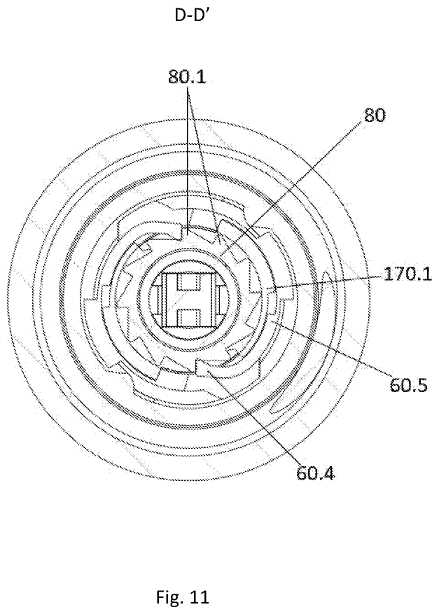

FIG. 11 shows a cross-sectional view along a plane D-D′ indicated in FIG. 9, of the second embodiment of the injection device according to the invention;

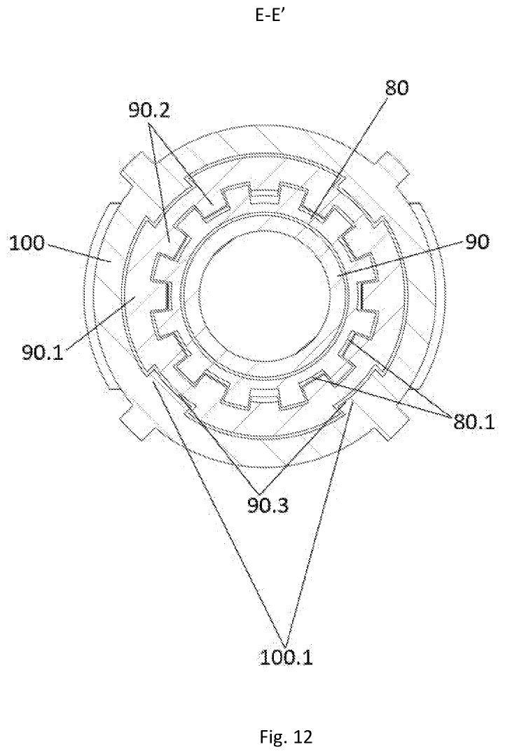

FIG. 12 shows a cross-sectional view along a plane E-E′ indicated in FIG. 9, of the second embodiment of the injection device according to the invention;

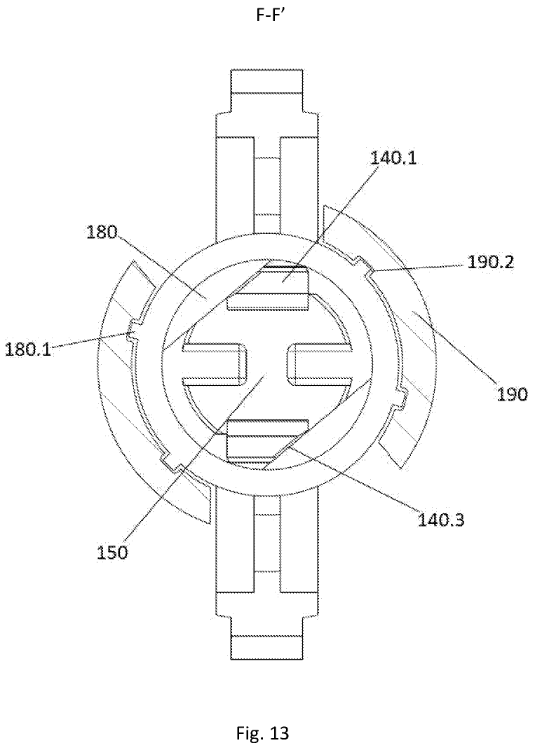

FIG. 13 shows a cross-sectional view along a plane F-F′ indicated in FIG. 9, of the second embodiment of the injection device according to the invention;

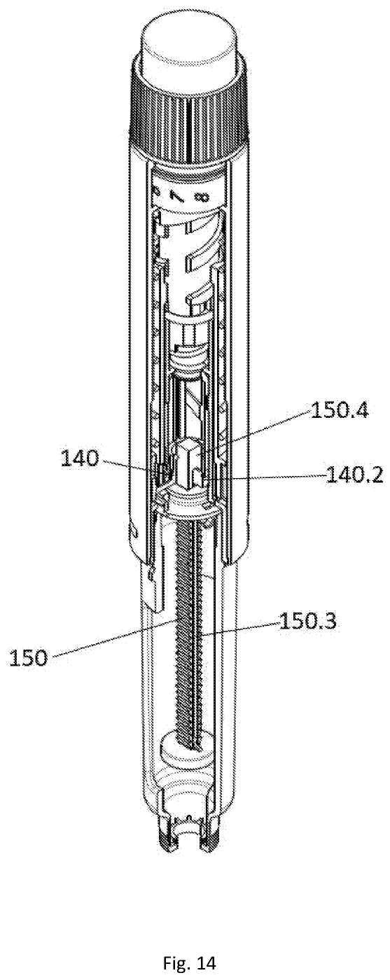

FIG. 14 shows a partial axonometric view of the second embodiment of the injection device according to the invention, showing a last dose blocking mechanism.

The injection device according to the invention shown in the figures has a longitudinal axis X-X along which its components are arranged. In the following description the term “proximal” refers to the end of the device where a button and a dose knob are located, while the term “distal” refers to the to the end thereof where a medicament cartridge is located and where a needle is to be installed.

FIG. 1-7 show the first embodiment of the invention.

The injection device shown in FIGS. 1-7 comprises a substantially cylindrical housing 1 and a cartridge holder 2 connected therewith, the cartridge holder being adapted to receive a cartridge 3 with a medicament (see FIGS. 2 and 3). A piston 3.1 is located in the cartridge 3. At a distal end of the cartridge holder 2 a thread 2.1 may be provided for mounting a needle module (not shown), through which the medicament is delivered. Alternatively, another state of art connection may be used enabling connection of the cartridge holder 2 with the needle module.

At a proximal end of the housing 1 of the injection device a rotational dose knob 4 and a button 5 are located. The dose knob 4 is a rotational element that may not be displaced axially.

As shown in FIG. 2, the dose knob 4 has projections ending with hook-like tabs 4.1 resting on a flange 1.1 located on the internal surface of the housing 1. The external surface of the dose knob 4 is designed to be grabbed by a user and it may comprise longitudinal grooves or other structures formed thereon facilitating handling and rotation of the dose knob 4. Within the housing 1 of the device, an indication window 1.2 may be provided through which the volume of a currently set dose is visible. This volume, indicated by an indication element located inside the housing 1, may also be visible through a transparent part of the housing 1 located above a scale provide on the indication element.

As shown in FIGS. 1, 2 and 3, the device according to the first embodiment of the invention further comprises a dose setting sleeve 6 which is blocked against axial movement, the dose setting sleeve 6 being rotated during dose setting, as well as a driving element 7 axially slidable and blocked against rotation within the housing 1.

The dose setting sleeve 6 has a thread 6.1 on its external surface, while the driving element 7 has an internal thread 7.1 cooperating with the external thread 6.1 of the dose setting sleeve 6. Alternatively, the dose setting sleeve 6 may have an internal thread and the driving element 7 may have an external thread. In this way the driving element 7 is coupled in rotation with the dose setting sleeve 6 and is displaced axially when the dose setting sleeve 6 is rotated.

In this first embodiment the dose setting sleeve 6 is coupled in rotation with the dose knob 4 by means of a multi-spline connection. The dose setting sleeve 6 has on its proximal side a widened part 6.2 resting at its distal side on a flange 1.3 formed in the housing 1, on the proximal side the widened part 6.2 being blocked against axial movement because it rests on the internal side of the dose knob 4.

On the external surface of the dose setting sleeve 6 a scale 6.3 may be printed, the scale 6.3 comprising the numbers indicating information about the volume the a currently set dose. The scale may be printed on at least a part of the circumference of the dose setting sleeve 6, in particular on the part of the external circumference where the thread 6.1 is formed.

In FIG. 4, presenting the cross-sectional view of the device along a plane A-A′ indicated in FIG. 2, it may be seen that the dose setting sleeve 6 has ratchet arms 6.4 (also shown in FIG. 1) at its distal end. There may be at least one ratchet arm 6.4 and in the described embodiment there are two of them. The ratchet arms 6.4 cooperate with a toothed gear 8 having a flange comprising circumferentially arranged internal teeth 8.1 cooperating with the ratchet arms 6.4 of the dose setting sleeve 6. The function of the ratchet arms 6.4 will be described in the following description.

The button 5 is connected with a button sleeve 9 which is axially engaged therewith, e.g. by a latch connection. The button sleeve 9 is a longitudinal tubular element provided with an external annular part 9.1 fixedly connected with the button sleeve 9. The button sleeve 9 may be connected with the button 5 through an intermediate element or it may be integral with the button 5. On the internal surface of the annular part 9.1 longitudinal projections 9.2 are formed constituting a multi-spline cooperating with the toothed gear 8 through its longitudinal grooves 8.2 shown in FIG. 5. When the button 5 is not pressed the toothed gear 8 is rotationally engaged with the button sleeve 9, while upon pressing the button 5 and displacing the button sleeve 9 in the distal direction, the projections 9.2 leave the grooves 8.2 of the toothed gear 8 allowing the toothed gear 8 to rotate. The toothed gear 8 is axially immobilized because it abuts an auxiliary sleeve 10 on one side and the dose setting sleeve 6 on the other side.

The auxiliary sleeve 10 is a stationary element blocked against rotation within the driving element 7, the blocking being realized e.g. by a multi-spline connection. The auxiliary sleeve 10 if further engaged on its proximal side with the button sleeve 9. The sliding connection of the auxiliary sleeve 10 with the button sleeve 9 is realized by longitudinal projections 10.1 of the auxiliary sleeve 10 cooperating with the longitudinal grooves 9.3 formed on the external surface of the annular part 9.1 of the button sleeve 9. The auxiliary sleeve 10 abuts on its distal side a non-translatable piston rod guide 11 or alternatively on another axially non-translatable element. The piston rod guide 11 will be described in the following description.

FIG. 5 shows the cross-sectional view of the device according to the first embodiment of the invention, along a plane B-B′ indicated in FIG. 2. In this figure multi-spline connections are visible between the auxiliary sleeve 10, the annular part 9.1 of the button sleeve 9 and the toothed gear 8. The button sleeve 9 is visible inside.

The connection of the button sleeve 9 with the toothed gear 8 and the auxiliary sleeve 10 may be realized in another way known to a man skilled in the art, so as to ensure mutual translation of these elements in the axial direction and to block their mutual rotation when the button 5 is not pressed.

The button sleeve 9 has also a surface 9.4 located on the annular part 9.1 and facing distally, the annular part 9.1 being adapted to interact with a coupling spring 12. The coupling spring 12 is located between said surface 9.4, and a ledge 10.2 having a form of a peripheral flange of the auxiliary sleeve 10. When the button 5 is not pressed, the coupling spring 12 may be loose, i.e. not compressed or it may be partly compressed, so as to be able to be further compressed under the action of the button 5 via the button sleeve 9.

Returning now to FIGS. 1, 2 and 3, they show the driving element 7. It has a form of a sleeve having, at its distal end, a widened part 7.2 against which a driving spring 13 leans.

The driving spring 13 is designed for storing the energy necessary for injection of a dose of medicament. As mentioned above, the driving spring 13 leans on its distal side against the widened part 7.2 of the driving element 7. On the proximal side, the driving spring 13 leans against the flange 1.3 of the housing 1. In the described embodiment, the driving spring 13 is a helical compression spring, but alternatively another linearly-deforming elastic energy storing element may also be used. The driving spring 13 may also be fixed to at least one additional element which is fixedly fitted in the housing 1 or coupled with the driving element 7. Alternatively, the driving spring 13 may lean against the dose setting sleeve 6.

On the internal surface of the driving element 7 driving arms 14 are fixed. They are stationary in the axial direction in relation to the driving element 7, optionally they may constitute an integral part of the driving element 7.

The device according to the first embodiment has also a piston rod 15 shown in FIGS. 1, 2, 3 and 6. The piston rod 15 has a non-circular cross-section and it is provided on its external surface with a least one row of ratchet teeth 15.1 designed for engagement of the driving element 7. In the present embodiment, said engagement is realized by the driving arms 14 having projections 14.1 interacting with the ratchet teeth 15.1, as shown in FIG. 6. The ratchet teeth 15.1 are located on the internal surface of the piston rod 15 and they are also coupled with ratchet arms 11.1 of the piston rod guide 11 which, in this embodiment, is mounted fixedly in the housing 1 by means of a locking member 16. Alternatively, the piston rod guide 11 may be mounted directly in the housing 1 or it may constitute an integral part thereof. Due to the locking member 16 the movement of the piston rod is blocked in the proximal direction, while it can be moved in the distal direction. The ratchet teeth 15.1 of the piston rod 15 may interact both with the driving arms 14 and with the piston rod guide 11. The piston rod guide 11 is provided with an opening having a shape corresponding to the non-circular cross-section of the piston rod 15 sliding therethrough. The distal end of the piston rod 15 has a piston rod ending 15.2 fixed thereon, the ending 15.2 increasing the contact surface between the piston rod 15 and a piston 3.1. Optionally, the piston rod 15 and its ending may constitute a single integral element.

The device according to the first embodiment may also be equipped with a last dose blocking mechanism as shown in FIG. 7. The last dose blocking mechanism prevents setting a dose that is larger than the volume remaining in the cartridge 3. The blocking is realized by a longitudinal groove 15.3 formed in the piston rod 15 and by a widened part 15.4 formed at the end of the piston rod 15.

In the groove a guide 14.2 of the driving arms 14 is received, the guide 14.2 being adapted to slide within the groove until it abuts the widened part 15.4.

The driving arms 14 and the guide 14.2, together with the driving element 7, are moved axially in relation to the piston rod 15 during dose setting. Their displacement corresponds to the volume of the set dose. During injection of the dose the driving arms 14, the guide 14.2 and the piston rod 15 are moved together so that their mutual positions do not change.

Now the operation of the device according to the first embodiment will be described.

In order to set a dose a user rotates the dose knob 4 in a first rotation direction. The dose setting sleeve 6, being coupled with the dose knob 4 by a multi-spline, rotates with the dose knob 4. The ratchet arms 6.4 of the dose setting sleeve 6 are displaced to a next position on the flange of the toothed gear 8. During dose setting the toothed gear 8 is blocked against rotation because it is coupled with the button sleeve 9 which in turn is coupled with the auxiliary sleeve 10. The auxiliary sleeve 10 is engaged with the driving element 7 which is blocked against rotation in the housing 1. The maximal dose to be set may be defined by blocking means that may be located on the dose setting sleeve 6 and on the housing 1. Said blocking means may be formed as cooperating projections or they may have a different shape selected by a man skilled in the art. The blocking means may also be arranged in a way enabling to select only one defined volume of a dose.

The rotation of the dose setting sleeve 6 in the setting direction causes the driving element 7 to be axially displaced in the proximal direction because these elements are engaged by means of a thread. Translation of the driving element 7 causes compression of the driving spring 13 so that the energy to be used for injection is stored therein. The driving arms 14 are displaced axially with the driving element 7, the projections 14.1 of the driving arms 14 snapping into subsequent positions on the ratchet teeth 15.1 of the piston rod 15.

In order to deliver the set dose the user presses the button 5 causing the button sleeve 9 to be displaced in the distal direction because these two elements are axially coupled. As a result of this displacement the connection between the button sleeve 9 and the toothed gear 8 becomes disconnected. The mechanism is not locked then and the driving spring 13 may release the stored energy. Hence, the driving spring 13 presses against the driving element 7 causing it to be translated in the distal direction. Together with the driving element 7 the driving arms 14 are moved pressing the piston rod 15 which in turn presses against the piston 3.1 via the ending 15.2. The pressed piston 3.1 located in the cartridge 3 with medicament causes the required dose of medicament to be pushed out of the cartridge 3.

The translation of the driving element 7 causes rotation of the dose setting sleeve 6 which returns to its initial position. When the button 5 is pressed, the translation of the button sleeve 9 causes the coupling spring 12 to be compressed. Upon release of the button 5, the coupling spring 12 is being extended. By releasing the stored energy the coupling spring 12 presses against the button sleeve 9 and makes it return to its initial position together with the button 5. The button sleeve 9 gets coupled with the toothed gear 8 again and the device is back in a state ready for setting a next dose of medicament to be injected.

FIGS. 8-14 show a second embodiment in which the required dose may be both set and corrected.

The device shown in FIGS. 8-14 comprises—similarly to the first embodiment—a substantially cylindrical housing 1 and a cartridge holder 20 connected therewith, the cartridge holder being adapted to receive a cartridge 30 with a medicament (see FIGS. 92 and 10). At a distal end of the cartridge holder 20 a thread 20.1 may be provided for mounting a needle module (not shown), through which the medicament is delivered. Alternatively, another state of art connection may be used enabling connection of the cartridge holder 20 with the needle module.

At a proximal end of the housing 1 of the injection device a rotational dose knob 40 and a button 50 are located. The dose knob 40 is designed to enable selection of a dose of medicament. It is a rotational element that may not be displaced axially.

As shown in FIG. 9, the dose knob 40 has projections ending with hook-like tabs 40.1 resting on a flange 1.1 located on an internal surface of the housing 1. The external surface of the dose knob 40 is designed to be grabbed by a user and it may comprise longitudinal grooves or other structures formed thereon facilitating handling and rotation of the dose knob 40. Within the housing 1 of the device, an indication window 1.2 may be provided through which the volume of a currently set dose is visible. This volume, indicated by an indication element located inside the housing 1, may also be visible through a transparent part of the housing 1 located above a scale provide on the indication element.

As shown in FIGS. 8, 9 and 10, the device according to the second embodiment of the invention further comprises a dose setting sleeve 60 which is blocked against axial movement, the dose setting sleeve being rotated during dose setting and dose correcting, as well as a driving element 70 axially slidable and blocked against rotation within the housing 1.

The dose setting sleeve 60 has a thread 60.1 on its external surface, while the driving element 70 has an internal thread 70.1 cooperating with the external thread 60.1 of the dose setting sleeve 60. Alternatively, the dose setting sleeve 60 may have an internal thread and the driving element 70 may have an external thread. In this way the driving element 70 is coupled in rotation with the dose setting sleeve 60 and is displaced axially when the dose setting sleeve 60 is rotated.

On the internal surface of the driving element 70 driving arms 140 are attached. They are immobilized in the axial direction in relation to the driving element 70, optionally they may constitute an integral part of the driving element 70. In this embodiment at least some of the driving arms 140 may be moved in radial direction so as their distance from the longitudinal axis X-X of the injection device may be changed.

In this second embodiment the dose setting sleeve 60 has on its proximal side a widened part 60.2 resting on its distal side on a flange 1.3 formed in the housing 1, on the proximal side the widened part 60.2 being blocked against axial movement because it rests on the internal side of the dose knob 40.

On the external surface of the dose setting sleeve 60 a scale 60.3 may be printed, the scale 60.3 comprising the numbers indicating information about the volume the a currently set dose. The scale may be printed on at least a part of the circumference of the dose setting sleeve 60, in particular on the part of the external circumference where the thread 60.1 is formed.

In FIG. 11, presenting the cross-sectional view of the device along a plane D-D′ indicated in FIG. 9, it may be seen that the dose setting sleeve 60 has ratchet arms 60.4 (also shown in FIG. 8) at its distal end. There may be at least one ratchet arm 60.4 and in the described embodiment there are two of them.

The ratchet arms 60.4 cooperate with a toothed gear 80 having a flange comprising circumferentially arranged internal teeth 80.1 cooperating with the ratchet arms 60.4 of the dose setting sleeve 60. The function of the ratchet arms 60.4 will be described in the following description.

The device according to the second embodiment is further provided with a coupling element 170 having a form of a hollow sleeve located between the dose setting sleeve 60 and a button sleeve 90.

The coupling element 170 is at its proximal end rotationally coupled with the button 50. The coupling may be realized as a multi-spline connection. At its distal end the coupling element 170 has two coupling arms 170.1 located between the ratchet arms 60.4 of the dose setting sleeve 60 and the teeth 80.1 of the toothed gear 80. In this embodiment the device may have at least one coupling arm 170.1. The number of the coupling arms 170.1 may correspond to the number of the ratchet arms 60.4 located at the distal end of the dose setting sleeve 60.

Besides the ratchet arms 60.4, the dose setting sleeve 60 has at its distal end two driving projections 60.5. The dose setting sleeve 60 may have at least one driving projection 60.5. The number of the ratchet arms 60.4 may be equal to the number of the driving projections 60.5. The ratchet arms 60.4 and the driving projections 60.5 are formed in such a way that the coupling arms 170.1 of the coupling element 170 may lean on one side on the driving projections 60.5 so as to block mutual rotation of the coupling element 170 and the dose setting sleeve 60. As a consequence, the rotation of the coupling element 170 (caused by the rotation of the dose knob) causes rotation of the dose setting sleeve 60. The relation between the ratchet arms 60.4, the driving projections 60.5 and the coupling arms 170.1 is shown in FIG. 11.

The button 50 is connected with a button sleeve 90 which is axially engaged therewith, e.g. by a latch connection. The button sleeve 90 is a longitudinal tubular element provided with an external annular part 90.1 fixedly connected with the button sleeve 90. The button sleeve 90 may be connected with the button 50 through an intermediate element or it may be integral with the button 50. On the internal surface of the annular part 90.1 longitudinal projections 90.2 are formed constituting a multi-spline cooperating with the toothed gear 80 through its longitudinal indents 80.2 shown in FIG. 12. When the button 50 is not pressed, the toothed gear 80 is rotationally engaged with the button sleeve 90, while upon pressing the button 50 and displacing the button sleeve 90 in the distal direction, the projections 90.2 leave the toothed gear 80 allowing it to rotate. The toothed gear 80 is axially immobilized because it abuts an auxiliary sleeve 100 on one side and the dose setting sleeve 60 on the other side.

The auxiliary sleeve 100 is a stationary element blocked against rotation within the driving element 70, the blocking being realized e.g. by a multi-spline connection. The auxiliary sleeve 100 if further engaged on its proximal side with the button sleeve 90. The sliding connection of the auxiliary sleeve 100 with the button sleeve 90 is realized by longitudinal projections 100.1 of the auxiliary sleeve 100 cooperating with the longitudinal grooves 90.3 formed on the external surface of the annular part 90.1 of the button sleeve 90. The auxiliary sleeve 100 abuts on its distal side a non-translatable piston rod guide 110 or alternatively on another axially non-translatable element. The piston rod guide 110 will be described in the following description.

FIG. 12 shows the cross-sectional view of the device according to the second embodiment of the invention, along a plane E-E′ indicated in FIG. 9. In FIG. 12 multi-spline connections are visible between the auxiliary sleeve 100, the annular part 90.1 of the button sleeve 90 and the toothed gear 80. The button sleeve 90 is visible inside.

The connection of the button sleeve 90 with the toothed gear 80 and the auxiliary sleeve 100 may be realized in another way known to a man skilled in the art, so as to ensure mutual translation of these elements in the axial direction and to block their mutual rotation when the button 50 is not pressed.

The button sleeve 90 has also a surface 90.4 located on the annular part 90.1 and facing distally, the annular part 90.1 being adapted to interact with a coupling spring 120. The coupling spring 120 is located between said surface 90.4 and a ledge 100.2 having a form of a peripheral flange of the auxiliary sleeve 100. When the button 50 is not pressed, the coupling spring 120 may be loose, i.e. not compressed or it may be partly compressed, so as to be able to be further compressed under the action of the button 50 via the button sleeve 90.

Returning now to FIGS. 8, 9 and 10, they show the driving element 70. It has a form of a sleeve having, at its distal end, a widened part 70.2 against which a driving spring 130 leans.

The driving spring 130 is designed for storing the energy necessary for injection of a dose of medicament. As mentioned above, the driving spring 130 leans, on its distal side, against the widened part 70.2 of the driving element 70. On the proximal side, the driving spring 130 leans against the flange 1.3 of the housing 1. In the described embodiment, the driving spring 130 is a helical compression spring, but alternatively another linearly-deforming elastic energy storing element may also be used. The driving spring 130 may also be fixed to at least one additional element which is fixedly fitted in the housing 1 or coupled with the driving element 70. Alternatively, the driving spring 130 may lean against the dose setting sleeve 60.

The device according to the second embodiment has also a piston rod 150 shown in FIGS. 8, 9, 10 and 13. The piston rod 150 has a non-circular cross-section and it is provided, on its external surface with a least one row of ratchet teeth 150.1 designed for engagement of the driving element 70. In the present embodiment, said engagement is realized by the driving arms 140 having projections 140.1 interacting with the ratchet teeth 150.1, as shown in FIG. 13 presenting a cross-section along a line F-F′ indicated in FIG. 9. As mentioned above, the driving arms 140 are attached to the internal surface of the driving element 70 and they are axially immobilized in relation to the driving element 70. However, in this second embodiment, at least some of the driving arms 140 may be moved in radial direction. The distal end of the piston rod 150 has a piston rod ending 150.2 fixed thereon, the ending 150.2 increasing the contact surface between the piston rod 150 and a piston 30.1. Optionally, the piston rod 150 and its ending may be formed as a single integral element.

In this embodiment, the possibility to move the driving arms 140 in the radial direction is realized by a cam element 180 and a cam sleeve 190. The cam element 180 and the cam sleeve 190 are adapted to be able to displace the driving arms 140 in the radial direction. The cam sleeve 190 is located within the auxiliary sleeve 100 and it is axially coupled at its proximal end, in a way enabling mutual rotation, with the distal end of the button sleeve 90, for example by a latch connection. The cam sleeve 190 has a spiral groove 190.1 on its external surface, the spiral groove 190.1 interacting with a projection 100.3 formed inside the stationary auxiliary sleeve 100. The cam element 180 is located inside the cam sleeve 190, the cam element 180 being coupled with the cam sleeve 190 rotationally but in a way enabling axial movement. This coupling is realized by means of longitudinal indents 190.2 located inside the cam sleeve 190 and longitudinal protrusions 180.1 of the cam element 180 facing outwards as shown in FIG. 13. Due to this coupling, when the button sleeve 90 is translated, the cam sleeve 190 is moved along a spiral path, upwards or downwards depending on the direction of translation of the button sleeve 90. The cam element 180 located within the cam sleeve 190 is axially engaged with the driving arms 140, e.g. by a latch connection.

As shown in FIG. 13, the driving arms 140 have slanted surfaces 140.3 formed on the external surfaces of the projections 140.1. The slanted surfaces 140.3 cooperate with the internal surface of the cam element 180. Due to the fact that the cam sleeve 190 may be rotated in relation to the button sleeve but is rotationally blocked in relation to the cam element 180, during the spiral movement of the cam sleeve 190 the cam element 180 also rotates clamping the driving arms 140 on the teeth 150.1 of the piston rod 150 or moving the driving arms 140 away from the ratchet teeth 150.1. Due to this solution the engagement of the driving arms 140 with the piston rod 150 may be releaseable.

The ratchet teeth 150.1 located on the external surface of the piston rod 150 are also engaged with ratchet teeth 110.1 of the piston rod guide 110. In this embodiment the piston rod guide 110 is fixedly mounted directly in the housing. Alternatively, the piston rod guide 110 may be mounted in the housing via an additional element or it may constitute an integral part of the housing. As a consequence, the movement of the piston rod 150 in the proximal direction is blocked while it may be displaced in the distal direction. The same ratchet teeth 150.1 of the piston rod 150 may cooperate both in the driving arms 140 and with the piston rod guide 110. In the piston rod guide 110 there is an opening having a shape corresponding to the non-circular cross-section of the piston rod 150 sliding therethrough.

The device according to the second embodiment may be provided with a last dose blocking mechanism as shown in FIG. 14.

The last dose blocking mechanism prevents setting a dose that is larger than the amount of medicament remaining in the cartridge 30. The blocking function is realized by means of a longitudinal groove 150.3 formed in the piston rod 150, the groove 150.3 having a widened part 150.4 at its end. A guide 140.2 of the driving arms 140 is received in the groove 150.3, the guide 140.2 being axially movable up to the point where it abuts the widened part.

During dose setting and dose correcting, the driving arms 140 with the guide 140.2 are moved axially (together with the driving element 70) in relation to the piston rod 150 and their displacement corresponds to the volume of a set dose. During dose injection, the driving arms 140, the guide 140.2 and the piston rod 150 are displaced together so that their mutual positions do not change.

In the following, the operation of the device according to the second embodiment will be described.

In order to set a dose, a user rotates the dose knob 40 in a first rotational direction. The coupling element 170, engaged with the dose knob 40 by means of a multi-spline, rotates with the dose knob 40. The coupling arms 170.1 of the coupling element 170 press against the driving projections 60.5 of the dose setting sleeve 60 causing it to rotate in the dose setting direction. The ratchet arms 60.4 of the dose setting sleeve 60 are displaced to a next position on the flange of the toothed gear 80. During dose setting the toothed gear 80 is blocked against rotation because it is coupled with the button sleeve 90 which in turn is coupled with the auxiliary sleeve 100, the auxiliary sleeve 100 being coupled with the driving element 70 which is rotationally blocked in the housing 1. The maximal dose to be set may be defined by blocking means located e.g. on the dose setting sleeve 60 and the housing. The blocking means may have a form of interacting protrusions or another form selected by a man skilled in the art. The blocking means may also be located in such a way that they enable to select just one defined dose volume.

The rotation of the dose setting sleeve 60 in the dose setting direction causes axial translation of the driving element 70 in the proximal direction because these elements are threadedly engaged. The translation of the driving element 70 causes compression of the driving spring 130 so that the energy to be used for injection is stored therein. The driving arms 140 together with the cam element 180 are displaced axially in the proximal direction together with the driving element 70. During dose setting, the driving arms 140 are not clamped on the teeth 150.1 of the piston rod 150.

If a dose too large is set by the user, it may be reduced by rotation of the dose knob 40 in a second rotational direction. The coupling element 170 rotates together with the dose knob 40, the coupling arms 170.1 of which tilt the ratchet arms 60.4 of the dose setting sleeve 60 as they rotate in the second direction, causing the ratchet arms 60.4 to be disengaged from the toothed gear 80. Upon said disengagement the mechanism is no longer blocked and the driving spring 130 may release a part of the stored energy. When the driving spring 130 extends, it presses against the driving element 70 and causes its displacement in the distal direction. The driving arms 140 and the cam element 80 are displaced together with the driving element 70. However, the piston rod 150 remains stationary as it is not pressed by the disengaged driving arms 140. Due to the threaded engagement of the driving element 70 with the dose setting sleeve 60, the displacement of the driving element 70 results in a corresponding rotation of the dose setting sleeve 60. The rotation of the dose setting sleeve 60 lasts until the driving projections 60.5 of the dose setting sleeve 60 abut again the coupling arms 170.1 of the coupling element 170. In this position the ratchet arms 60.4 of the dose setting sleeve 60 are no longer tilted by the coupling arms 170.1 of the coupling element 170 and they become engaged with the toothed gear 80 in a position retracted by one tooth. When the ratchet arms 60.4 are engaged with the toothed gear 80 the whole mechanism is blocked again because the toothed gear 80 does not rotate, neither during dose setting nor dose correcting.

In order to deliver the set dose, the user presses the button 50 and the button sleeve 90 with the cam sleeve 190 are axially displaced therewith as these elements are in an axial movement engagement. In a first phase of this movement the button sleeve 90 is still rotationally engaged with the toothed gear 80. The cam sleeve 190 is moved spirally because it is coupled with the auxiliary sleeve 100 via a spiral path 190.1. The angular displacement of the cam sleeve 190 is transferred to the cam element 180, the rotation of which results in a displacement of the projections 140.1 of the driving arms 140 towards the axis of the device due to the shape of the interacting surfaces of the cam element 180 and the slanted surfaces 140.3. In this position, the projections 140.1 are now clamped on the teeth 150.1 of the piston rod 150. When the button 50 is pressed further, the button sleeve 90 is further translated axially and the button sleeve 90 becomes disengaged from the toothed gear 80. The mechanism is not blocked then and the driving spring 130 may release the stored energy. The driving spring 130 presses against the driving element 70 and the driving arms 140 are moved together with the driving element 70. The driving arms 140 press against the piston rod 150 pressing in turn against the piston 30.1 via the ending 150.2 located in the cartridge 30, causing the required dose of the medicament to be pushed out.

The translation of the driving element 70 in the distal direction causes the dose setting sleeve 60 to be rotated back so that it returns to its initial position. When the button 50 is pressed, the translation of the button sleeve 90 causes the coupling spring 120 to be compressed. Upon release of the button 50, the coupling spring 120 is extended and it releases the stored energy pressing the button sleeve 90 and making it return to the initial position together with the button 50. The button sleeve 90 becomes engaged with the toothed gear 80 again and the rotation of the cam sleeve 190 with the cam element 180 makes the projections 140.1 of the driving arms 140 move away from the piston rod 150. The device is then ready for setting a next dose of medicament.

1. An injection device for injecting a liquid substance, having a longitudinal axis, a proximal end and a distal end, the injection device comprising a housing and a cartridge holder in which a cartridge with said substance is contained, the housing being provided on the proximal side with a rotatable dose knob and an axially translatable button, the housing comprising:
a dose setting sleeve adapted to be rotationally engaged with the dose knob,
a non-rotatable tubular driving element slidingly mounted in the housing, the driving element being engaged with the dose setting sleeve by a threaded connection,
a non-rotatable axially sliding piston rod adapted to press against a piston located in the cartridge,
a toothed gear comprising teeth,
a driving spring,
wherein the dose setting sleeve is equipped with at least one ratchet arm cooperating with the toothed gear, the piston rod having an external surface comprising at least one row of ratchet teeth, the driving element has a distal side fixedly connected with at least one driving arm cooperating with the ratchet teeth of the piston rod,
a non-rotatable and axially slidable button sleeve engaged on its proximal side with the button,
an immovable auxiliary sleeve located within the driving element, and
wherein the button sleeve is detachably and slidingly engaged with the toothed gear and the button sleeve is slidingly engaged with the auxiliary sleeve by a coupling spring which is compressed when the button sleeve is translated in the distal direction.

2. The injection device according to claim 1, wherein the dose setting sleeve is adapted to be engaged with the dose knob by a tubular coupling element located between the dose setting sleeve and the button sleeve, the tubular coupling element being rotationally engaged with the dose knob and having a distal end comprising at least one coupling arm cooperating with at least one ratchet arm of the dose setting sleeve; and
wherein the at least one driving arm of the driving element is movable in a direction perpendicular to the longitudinal axis of the device.

3. The injection device according to claim 2, wherein the button sleeve has a distal end mutually rotatably engaged with a cam sleeve, the cam sleeve cooperating with a cam element which is rotationally coupled with the cam sleeve, the cam element cooperating with at least one driving arm of the driving element so as to enable its/their displacement between a position of engagement with the ratchet teeth of the piston rod and a position of disengagement with the ratchet teeth of the piston rod.

4. The injection device according to claim 2, wherein the dose setting sleeve has a distal end comprising at least one driving projection configured to be abutted by at least one coupling arm of the coupling element, so as to cause rotation of the dose setting sleeve.

5. The injection device according to claim 1, further comprises a piston rod guide disposed at a distal end of the injection device configured to enable the piston rod to move in the distal direction and blocking the movement of the piston rod in the proximal direction.

6. The injection device according to claim 5, wherein a distal end of the auxiliary sleeve rests on the piston rod guide.

7. The injection device according to claim 1, wherein the driving element is blocked against rotation in the housing by a multi-spline.

8. The injection device according to claim 1, wherein the dose setting sleeve is blocked in rotation with the dose knob by a multi-spline.

9. The injection device according to claim 2, wherein the coupling element is blocked in rotation with the dose knob by a multi-spline.

10. The injection device according to claim 1, wherein the button sleeve is provided with an external annular part fixedly connected therewith, the annular part having a proximal side comprising an internal multi-spline enabling a sliding detachable engagement with the toothed gear, and a distal side comprising an external multi-spline enabling a sliding engagement with the auxiliary sleeve.

11. The injection device according to claim 2, wherein the at least one coupling arm comprises a number of coupling arms equal to a number of the at least one ratchet arm and a number of the driving projections (60.5) of the dose setting sleeve (6, 60).

12. The injection device according to claim 1, wherein the teeth of the toothed gear are arranged annularly.

13. The injection device according to claim 1, wherein the teeth of the toothed gear are mutually parallel.

14. The injection device according to claim 1, wherein the toothed gear is blocked against rotation during dose setting and is configured to rotate during dose delivery.

15. The injection device according to claim 1, wherein the driving spring is a compression spring.

16. The injection device according to claim 1, wherein the driving spring is a helical compression spring.
